(19) United States
(12) Patent Application Publication
(10) Pub. No.: US 2019/0090795 A1
(43) Pub. Date: Mar. 28, 2019

(54) PULSE OXIMETER WITH PLAYFUL HOUSING

(71) Applicant: Mettaventure, LLC, Norwalk, CT (US)

(72) Inventor: Wendy BOLLER, Norwalk, CT (US)

(21) Appl. No.: 16/114,742

(22) Filed: Aug. 28, 2018

Related U.S. Application Data
(60) Provisional application No. 62/550,740, filed on Aug. 28, 2017.

(51) Int. Cl. A61B 5/1455; A61B 5/00

(52) U.S. Cl. CPC A61B 5/14552; A61B 5/6826; A61B 2562/0233; A61B 5/684; A61B 5/6815

(57) ABSTRACT

A pulse oximeter is provided comprising a housing that includes three-dimensional surface elements on upper and lower sections of the housing that are configured to resemble an animal or an object. The pulse oximeter is configured to receive a finger or an ear of a user to measure pulse and oxygen saturation of the user, and the three-dimensional surface elements of the upper housing and lower housing are collectively configured to resemble a mouth and a face of the animal or object for receiving the finger or ear of the user into the pulse oximeter. The housing can be permanently attached to the pulse oximeter or removable from the pulse oximeter so as to be replaceable with an alternate housing having different three-dimensional surface elements resembling a different animal or object.

### CROSS-REFERENCE TO RELATED APPLICATIONS

This application claims the benefit of U.S. Provisional Application No. 62/550,740, filed Aug. 28, 2017, which is hereby incorporated by reference in its entirety.

### BACKGROUND OF THE INVENTION

A pulse oximeter measures oxygen saturation and pulse rate. It is a common, noninvasive medical device used widely by healthcare providers and by individuals tracking their own health. Pulse oximeters can be purchased at drugstores and through medical supply companies without a prescription.

Pulse oximetry is used to detect changes in blood oxygen level. People with conditions including asthma, pneumonia, congestive heart failure, and chronic obstructive pulmonary disease use pulse oximeters. Pulse oximeters are also used regularly to take routine vital signs. Pulse oximeters are used in settings for wellness, critical care, and rehabilitation. Pulse oximetry is used in adult and pediatric healthcare settings.

A standard design pulse oximeter has a clip with a sensor to place over a finger. Another variation of pulse oximeter clips on the ear. Pulse oximeters come in a variety of colors, but with little variation of shape and design.

### SUMMARY OF THE INVENTION

The present invention provides an integrated or attachable housing or cover for a pulse oximeter device that is in the shape of an animal or other design having a mouth, or other design shape. The mouth portion of the housing, when appropriate to the specific design, is arranged on the end of the pulse oximeter that opens to receive the user's finger. The pulse oximeter of the present invention creates an opportunity to provide a moment of levity with patients to support healing and wellness.

In accordance with one embodiment of the present invention, a housing or a cover for a pulse oximeter is provided in the form of a face or mouth, or other stylized design, and the housing or cover is attached to an existing pulse oximeter.

In addition, the present invention may include a pulse oximeter that could have a “family” of clip on face/mouth housings or covers that can be substituted on and off of a single pulse oximeter.

The housing or cover of the present invention can be attached to an existing pulse oximeter in a number of manners, including: providing the stylized housing as an encasing structure similar to a phone case comprising two or more parts that snap together and fit over the existing pulse oximeter; providing the stylized housing as an animal shape or design with a mouth “skin” to fit over standard pulse oximeter; clipping the stylized housing onto a standard pulse oximeter; providing screws or bolts to screw or bolt the housing onto the pulse oximeter; gluing or otherwise affixing with adhesive the stylized housing onto a standard pulse oximeter; and providing the stylized housing as a soft pull on sock-like or glove-like case that can slide over an existing pulse oximeter, and is made of a flexible material such as neoprene or elastic.

The housing or cover of the present invention can be made from various materials, including for example silicone or any hard or soft plastic material.

In a further embodiment of the present invention, a new pulse oximeter mold can be provided with the face or mouth or other design shape integrally formed with the pulse oximeter, rather than attaching a housing or cover to a standard, existing pulse oximeter.

The size of the housing or casing may vary to fit particular sizes of pulse oximeters and may come in pediatric and adult sizes.

In additional embodiments of the present invention, the pulse oximeter and housing may be configured for use as an ear clip. The housing may also be configured to emit an audio sound, such as the sound of the animal that is presented in the housing design. In embodiments of the present invention in which the housing is in the design of an animal or other shape having eyes or eye-like elements, the eyes may be configured to light up. Alternative embodiments of the present invention may also provide a finger warming in the device or housing.

In accordance with the present invention, a pulse oximeter is provided comprising a pulse oximeter module configured to measure pulse and oxygen saturation and a housing for the pulse oximeter. The housing comprises an upper housing arranged on a top portion of the pulse oximeter module comprising three-dimensional surface elements configured to resemble a first portion of an animal or an object, and a lower housing arranged on a bottom portion of the pulse oximeter module comprising three-dimensional surface elements configured to resemble a second portion of the animal or the object.

In accordance with an embodiment of the pulse oximeter, the pulse oximeter module comprises a finger receiving portion configured to receive a finger of a user to measure pulse and oxygen saturation of the user, and the upper housing and lower housing define a housing finger receiving portion overlapping the pulse oximeter module finger receiving portion, and the three-dimensional surface elements of the upper housing and lower housing are collectively configured to resemble a mouth and a face of the animal or object for receiving the finger of the user into the pulse oximeter module finger receiving portion.

In accordance with a further embodiment of the pulse oximeter of the present invention, the upper housing is integrally formed with the top portion of the pulse oximeter module and the lower housing is integrally formed with the bottom portion of the pulse oximeter module. The upper housing may be integrally molded with the top portion of the pulse oximeter module and the lower housing may be integrally molded with the bottom portion of the pulse oximeter module. The pulse oximeter or pulse oximeter module may further comprise a display screen and an interface button.

In accordance with a still further embodiment of the pulse oximeter of the present invention, the upper housing is configured to be removably attached to the top portion of the pulse oximeter module and to at least partially cover the top portion of the pulse oximeter module, and the lower housing is configured to be removably attached to the bottom portion of the pulse oximeter module and to at least partially cover the bottom portion of the pulse oximeter module.

In one such embodiment of the pulse oximeter of the present invention in which the upper and lower housing are removable from the pulse oximeter module, the upper housing comprises an inner surface having a plurality of pins or dowels extending therefrom and a surface of the top portion of the pulse oximeter module comprises a plurality of holes configured to receive the plurality of pins or dowels of the upper housing to removably attach the upper housing to the top portion of the pulse oximeter module; and the lower housing comprises an inner surface having a plurality of pins or dowels extending therefrom and a surface of the bottom portion of the pulse oximeter module comprises a plurality of holes configured to receive the plurality of pins or dowels of the lower housing to removably attach the lower housing to the bottom portion of the pulse oximeter module.

In a further embodiment of the pulse oximeter of the present invention in which the upper and lower housing are removable from the pulse oximeter module, the upper housing comprises a locking tab and the top portion of the pulse oximeter module comprises a tab receiving portion configured to receive and engage the locking tab of the upper housing to removably attach the upper housing to the top portion of the pulse oximeter module; and the lower housing comprises a locking tab and the bottom portion of the pulse oximeter module comprises a tab receiving portion configured to receive and engage the locking tab of the lower housing to removably attach the lower housing to the bottom portion of the pulse oximeter module.

In a still further embodiment of the pulse oximeter of the present invention in which the upper and lower housing are removable from the pulse oximeter module, the top portion of the pulse oximeter module comprises a locking tab and the upper housing comprises a tab receiving portion configured to receive and engage the locking tab of the top portion of the pulse oximeter module to removably attach the upper housing to the top portion of the pulse oximeter module; and the bottom portion of the pulse oximeter module comprises a locking tab and the lower housing comprises a tab receiving portion configured to receive and engage the locking tab of the bottom portion of the pulse oximeter module to removably attach the lower housing to the bottom portion of the pulse oximeter module.

In a still further embodiment of the pulse oximeter of the present invention in which the upper and lower housing are removable from the pulse oximeter module, the upper housing and the top portion of the pulse oximeter module comprise overlapping openings configured to receive a screw or bolt to removably attach the upper housing to the top portion of the pulse oximeter module; and the lower housing and the bottom portion of the pulse oximeter module comprise further overlapping openings configured to receive a further screw or bolt to removably attach the lower housing to the bottom portion of the pulse oximeter module.

In a still further embodiment of the pulse oximeter of the present invention in which the upper and lower housing are removable from the pulse oximeter module, the upper housing is made from a flexible material and is configured to engage the top portion of the pulse oximeter module to removably attach the upper housing to the top portion of the pulse oximeter module; and the lower housing is made from a flexible material and is configured to engage the lower housing to removably attach the lower housing to the bottom portion of the pulse oximeter module.

In a still further embodiment of the pulse oximeter of the present invention in which the upper and lower housing are removable from the pulse oximeter module, wherein the upper housing comprises one or more clip or tab extending from the upper housing that is configured to clip the upper housing to the top portion of the pulse oximeter module to removably attach the upper housing to the top portion of the pulse oximeter module; and the lower housing comprises one or more clip or tab extending from the lower housing that is configured to clip the lower housing to the lower housing to removably attach the lower housing to the bottom portion of the pulse oximeter module.

In accordance with a further embodiment of the pulse oximeter of the present invention, the top portion of the pulse oximeter module comprises a top chassis and the bottom portion of the pulse oximeter module comprises a bottom chassis. The top chassis and the bottom chassis define a finger receiving portion therebetween configured to receive a finger of a user to measure pulse and oxygen saturation of the user, and the top chassis and the bottom chassis are attached by a plurality of pivots configured to enable the opening and closing of the pulse oximeter module to receive the finger of the user. The pulse oximeter module may further comprise a display screen, an interface button, and a bezel attached to the top chassis and configured to receive and house the display screen and the interface button. The bottom chassis may further comprise a battery holder configured to receive one or more batteries for supplying electric power to the pulse oximeter module and a removable battery cover configured to cover the battery holder. In accordance with one such embodiment, the upper housing comprises an inner surface having a plurality of pins or dowels extending therefrom and the bezel comprises a plurality of holes configured to receive the plurality of pins or dowels of the upper housing to removably attach the upper housing to the bezel and top chassis of the pulse oximeter module; and the lower housing comprises an inner surface having a plurality of pins or dowels extending therefrom and the bottom chassis and the battery cover of the pulse oximeter module comprise a plurality of holes configured to receive the plurality of pins or dowels of the lower housing to removably attach the lower housing to the bottom chassis of the pulse oximeter module.

In accordance with any of the above referenced embodiments of the pulse oximeter of the present invention, the three-dimensional surface elements of the upper housing and the three-dimensional surface elements of the lower housing are collectively configured to resemble an alligator, a fish, a shark, a dog, a car, a robot or a frog.

In accordance with any of the above referenced embodiments of the pulse oximeter of the present invention, the top portion of the pulse oximeter module and the bottom portion of the pulse oximeter module may be attached by a plurality of pivots configured to enable the opening and closing of the pulse oximeter to receive a finger or an ear of a user.

In a further embodiment of the pulse oximeter of the present invention, the pulse oximeter module comprises an ear receiving portion configured to receive an ear of a user to measure pulse and oxygen saturation of the user, and the upper housing and lower housing define a housing ear receiving portion overlapping the pulse oximeter module ear receiving portion, and the three-dimensional surface elements of the upper housing and lower housing are collectively configured to resemble a mouth and a face of the animal or object for receiving the ear of the user into the pulse oximeter module ear receiving portion.

In accordance with any of the above referenced embodiments of the pulse oximeter of the present invention, in a further embodiment of the pulse oximeter, the pulse oximeter module is configured to emit an audio tone upon opening of the pulse oximeter, and the audio tone corresponds to a sound relating to the animal or the object resembled by the housing.

In accordance with any of the above referenced embodiments of the pulse oximeter of the present invention, in a further embodiment of the pulse oximeter, the pulse oximeter comprises one or more lighting elements configured to emit a light through the three-dimensional surface elements of either or both of the upper housing and lower housing.

In a further embodiment of the pulse oximeter of the present invention, the pulse oximeter comprises a display screen and an interface button.

In a further embodiment of the pulse oximeter of the present invention, the pulse oximeter, wherein the pulse oximeter module comprises a receiving portion configured to receive a finger or an ear of a user to measure pulse and oxygen saturation of the user, and wherein the upper housing and lower housing define a housing receiving portion overlapping the pulse oximeter module receiving portion, and the three-dimensional surface elements of the upper housing and lower housing are collectively configured to resemble a mouth and a face of the animal or object for receiving the finger of the user into the pulse oximeter module finger receiving portion. In such embodiment, the upper housing may be integrally formed with the top portion of the pulse oximeter module and the lower housing may be integrally formed with the bottom portion of the pulse oximeter module. Alternatively, the upper housing can be configured to be removably attached to the top portion of the pulse oximeter module and to at least partially cover the top portion of the pulse oximeter module, and the lower housing configured to be removably attached to the bottom portion of the pulse oximeter module and to at least partially cover the bottom portion of the pulse oximeter module.

In a further embodiment of the pulse oximeter of the present invention, the pulse oximeter comprises a plurality of housings comprising a plurality of upper housings each configured to resemble a first portion of a different animal or object and a plurality of lower housings each configured to resemble a second portion of the different animal or object, and the plurality of housings are each removable from the pulse oximeter module and can be substituted for another one of the plurality of housings.

### BRIEF DESCRIPTION OF THE DRAWINGS

FIG. 1 shows a pulse oximeter in accordance with a first embodiment of the present invention having a housing in the shape of an alligator;

FIG. 2A shows a perspective view of a pulse oximeter in accordance with a second embodiment of the invention having a housing in the shape of a fish;

FIG. 2B shows a left side view of the pulse oximeter in accordance with the second embodiment of the invention;

FIG. 2C shows a top side view of the pulse oximeter in accordance with the second embodiment of the invention;

FIG. 2D shows a front side view of the pulse oximeter in accordance with the second embodiment of the invention;

FIG. 2E shows a rear side view of the pulse oximeter in accordance with the second embodiment of the invention;

FIG. 2F shows an exploded view of the pulse oximeter in accordance with the second embodiment of the invention;

FIG. 3A shows an exploded view of the pulse oximeter in accordance with the third embodiment of the invention;

FIG. 3B shows a further exploded view of the pulse oximeter in accordance with the third embodiment of the invention;

FIG. 3C shows an exploded view of a pulse oximeter module of the pulse oximeter in accordance with the third embodiment of the invention;

FIG. 3D shows a further exploded view of the pulse oximeter module of the pulse oximeter in accordance with the third embodiment of the invention;

FIG. 3E shows a view of the various components of the pulse oximeter in accordance with the third embodiment of the invention;

FIG. 4A shows a first perspective view of a pulse oximeter in accordance with a fourth embodiment of the invention having a housing in the shape of a shark;

FIG. 4B shows a second perspective view of the pulse oximeter in accordance with the fourth embodiment of the invention;

FIG. 4C shows a third perspective view of the pulse oximeter in accordance with the fourth embodiment of the invention;

FIG. 4D shows a perspective view of the pulse oximeter in an opened position in accordance with the fourth embodiment of the invention; and

FIGS. 5A-5I show various further embodiments of a pulse oximeter in accordance with the present invention, including pulse oximeters having housings in the shapes of a dog, fish, robot, car and frog.

### DETAILED DESCRIPTION OF THE INVENTION

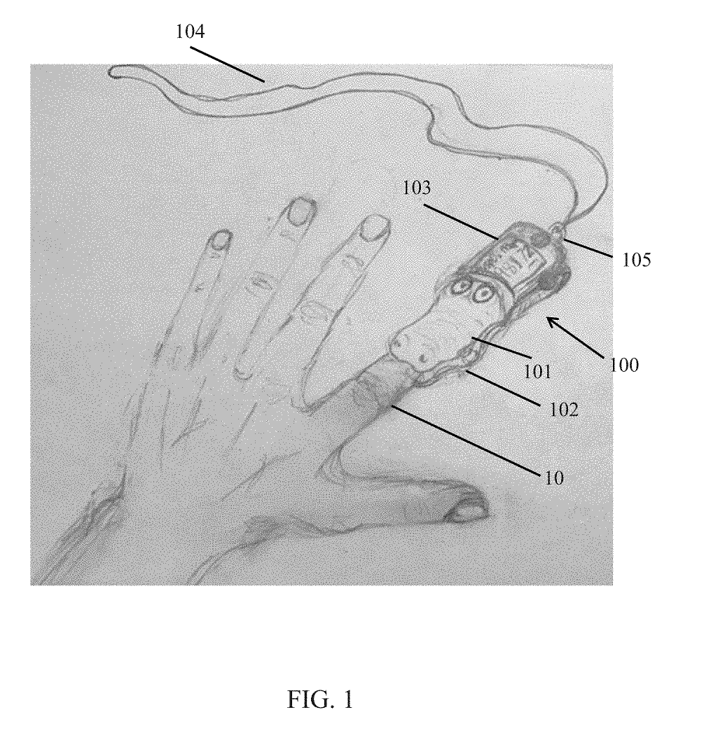

The present invention will now be described with reference made to FIGS. 1-5I

In accordance with the present invention, a specialized pulse oximeter and housing for a pulse oximeter is provided. As illustrated in the exemplary embodiment shown in FIG. 1, a pulse oximeter 100 is provided having a specialized housing 101 comprising surface elements configured to resemble an object, such as three-dimensional surface elements resembling an alligator. The pulse oximeter 100 is a pulse oximeter for use on a patient's finger 10, and the housing 101 comprises a finger receiving portion 102, into which the patient inserts his or her finger 10. The finger receiving portion 102 of the housing 101 comprises a three dimensional structure resembling the mouth of the alligator, such that when the patient inserts his or her finger 10 into the pulse oximeter 100 for use, the opening and closing of the pulse oximeter 100 resembles the opening and closing of the alligator's mouth. The housing 101 comprises an upper housing resembling the top of the mouth and a lower housing comprising resembling the bottom of the mouth. Although the pulse oximeter 100 shown in FIG. 1 comprises a housing 101 resembling an alligator, other embodiments of the pulse oximeter housing 101 of the present invention may take other forms.

The pulse oximeter 100 may comprise a screen 103 configured to display data measured by the pulse oximeter 100 and other information pertinent to the operation of the pulse oximeter 100, as known in the art. The pulse oximeter 100 may additionally or alternatively comprise a cord or string 104 attached to the pulse oximeter 100, such as through a loop 105 on the end of the pulse oximeter 100, to enable carrying the pulse oximeter 100 around one's neck.

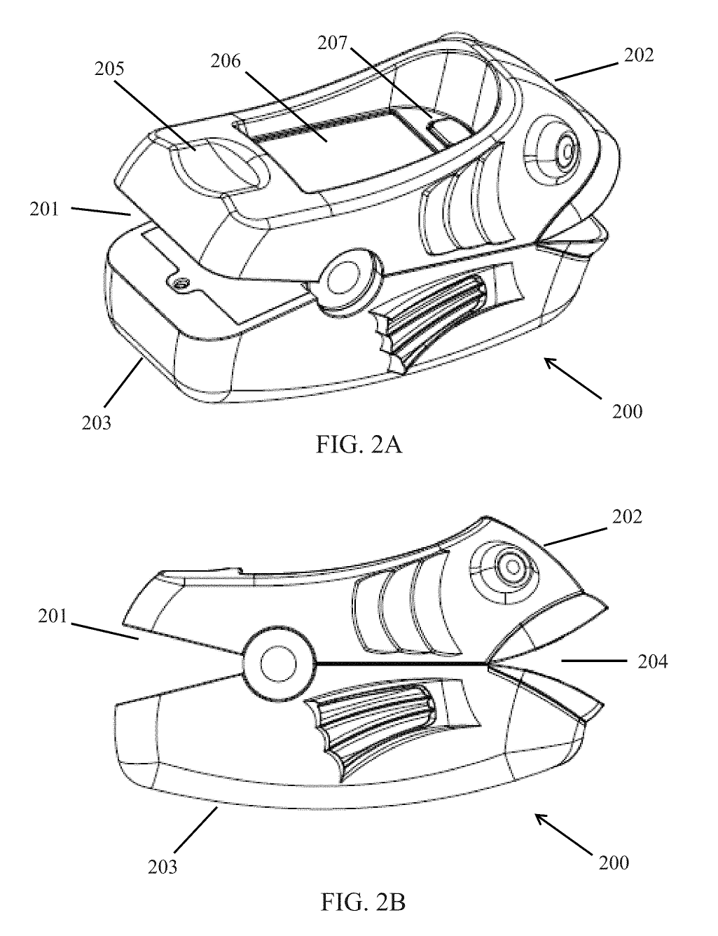

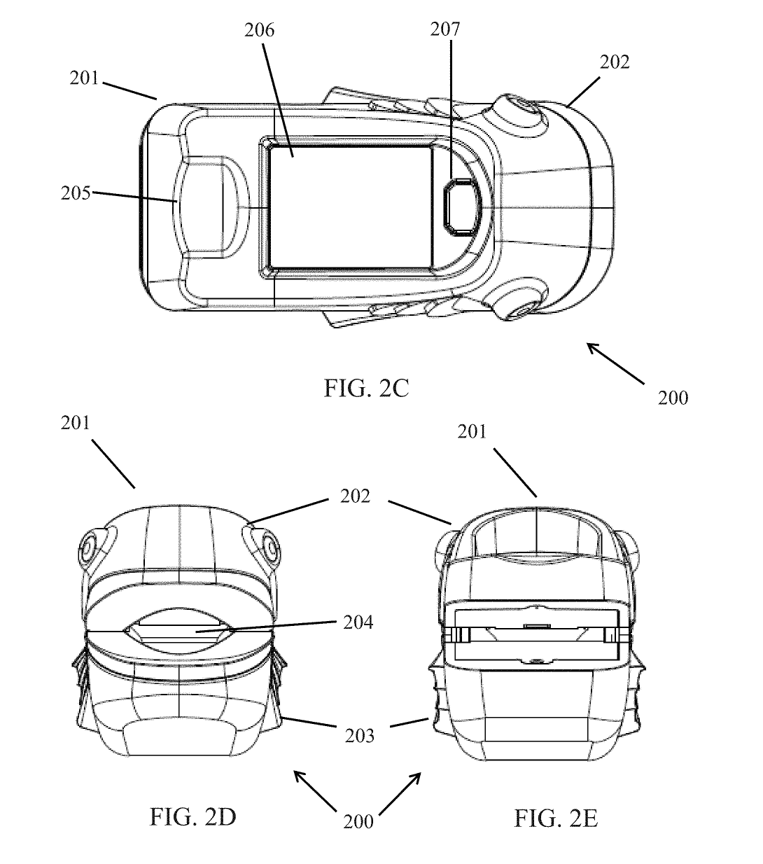

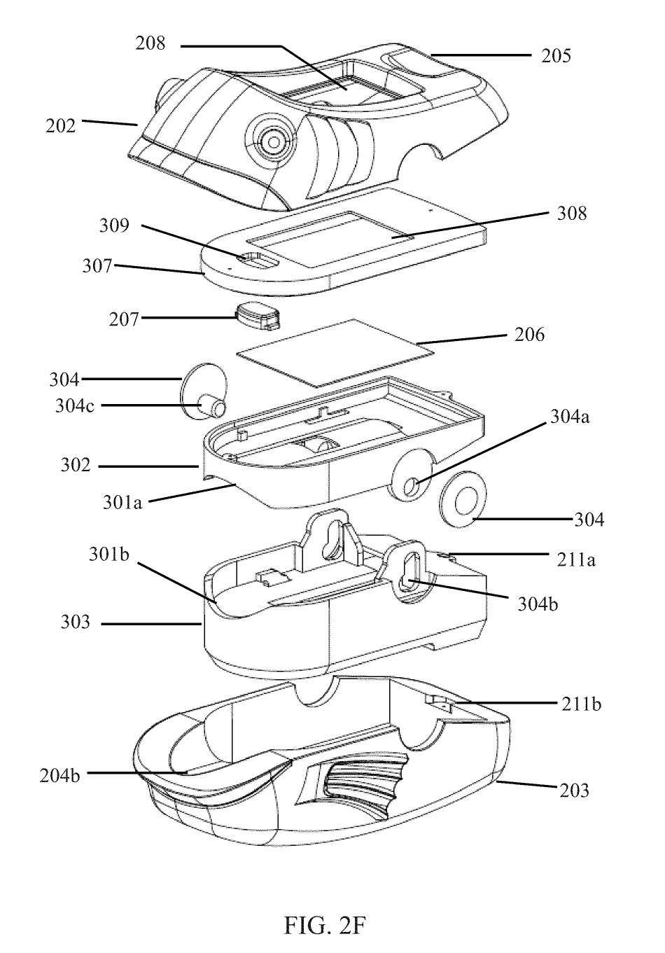

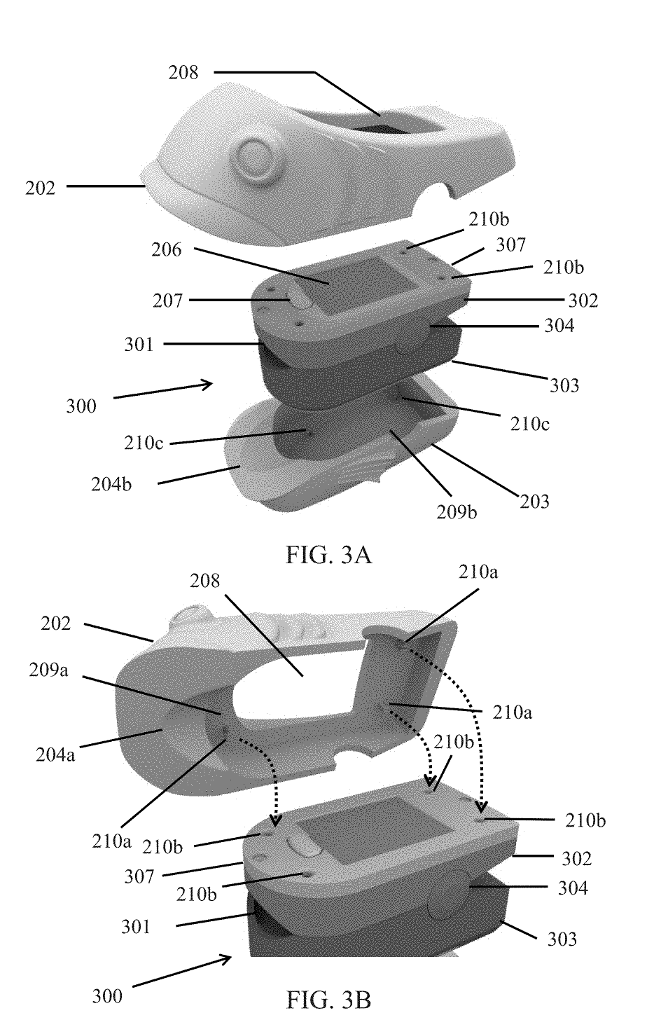

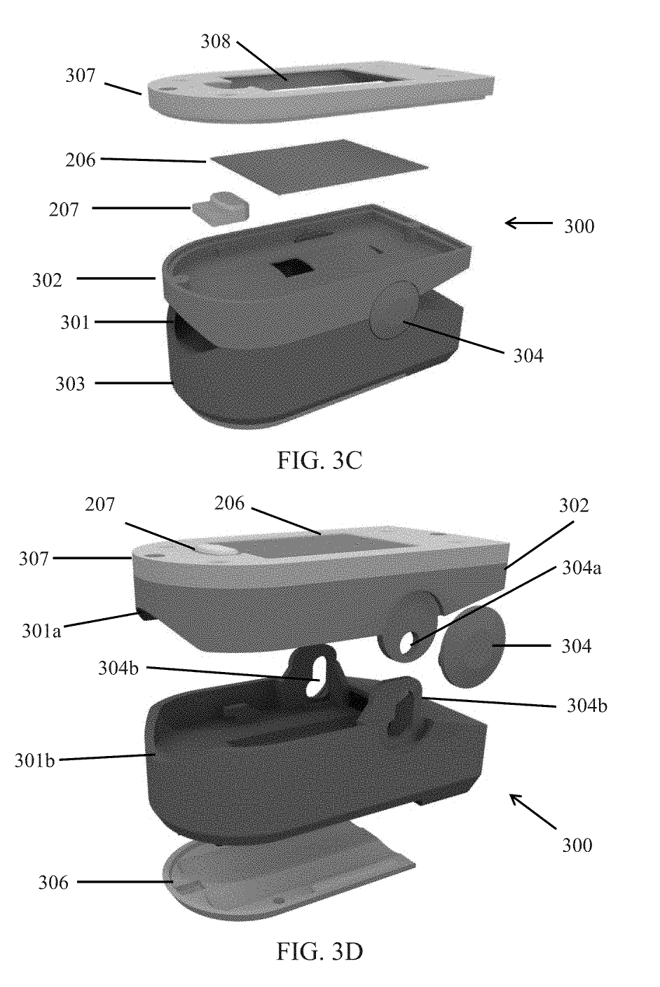

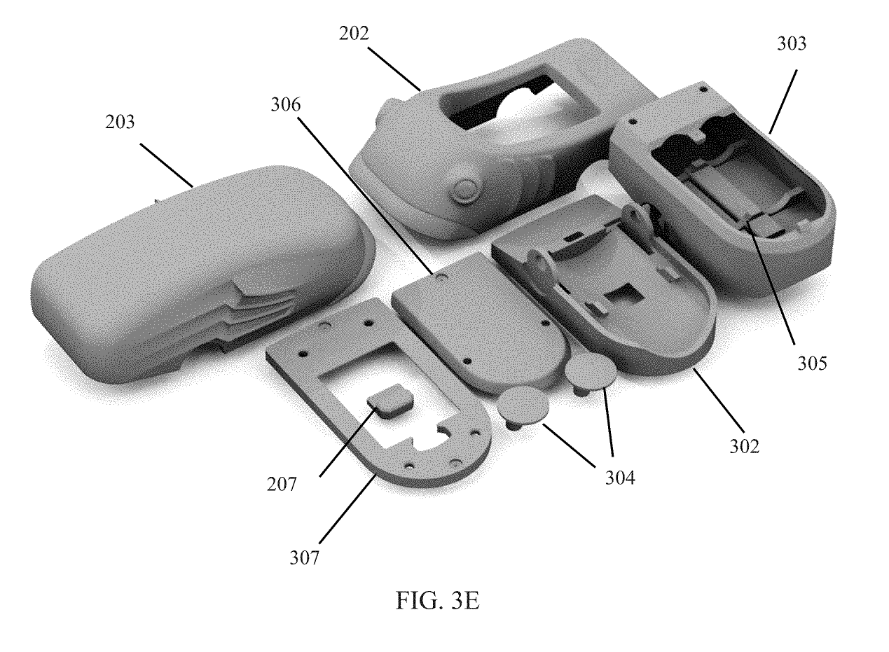

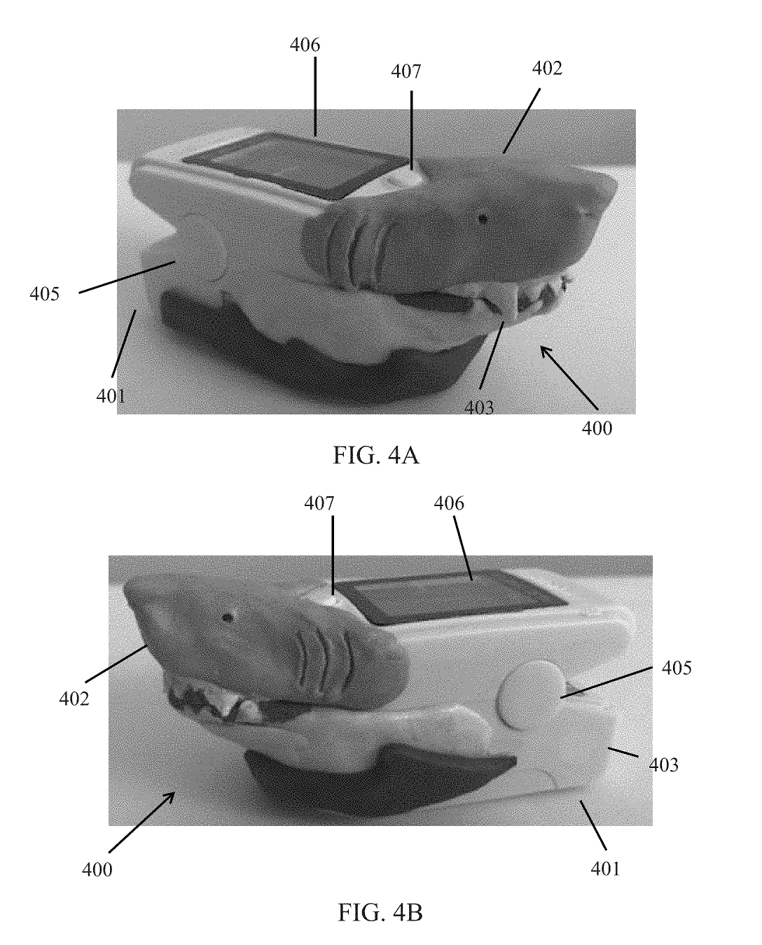

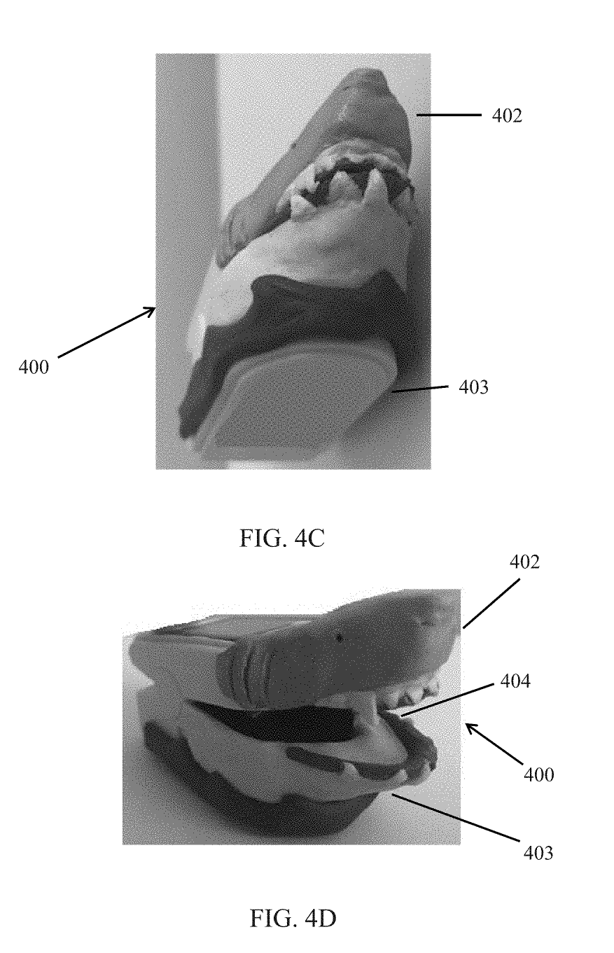

The pulse oximeter housing 101 of the present invention may be provided as a detachable upper and lower housing elements that are configured to serve as a removable cover for the pulse oximeter, such as a commercially available and separate pulse oximeter device, as shown in FIGS. 2A-3E. Alternatively, as shown in FIGS. 4A-4D, the pulse oximeter housing may be integrally formed with the pulse oximeter device.

FIGS. 2A-3E show further embodiments of the pulse oximeter 200 of the present invention, which comprises a housing 201 that can be attached and detached from the pulse oximeter module 300. A housing 201 is provided that covers the pulse oximeter module 300 to provide the pulse oximeter 200 with a stylized and playful appearance. In the embodiments shown in FIGS. 2A-3E, the upper housing 202 and lower housing 203 comprise three-dimensional surface elements resembling a fish, such as eyes, gills, fins and a mouth. Other designs may be provided in alternative embodiments without departing from the scope of the invention.

The housing 201 comprises an upper housing 202 covering the top of the pulse oximeter module 300 and a lower housing 203 covering the bottom of the pulse oximeter module 300. The upper and lower housing 202, 203 are arranged so as to form a finger receiving portion 204 aligned with the finger receiving portion 301 of the pulse oximeter module 300, into which the user can insert his or her finger in order to use the pulse oximeter 200. In the embodiments shown in FIGS. 2A-3E, the housing 201 is configured to provide the appearance of a fish, and the finger receiving portion 204 of the pulse oximeter 200 is configured to resemble the mouth of a fish. The upper housing 202 and lower housing 203 may each comprise a groove 204a, 204b to provide a space accommodating the user's finger when the user inserts his or her finger into the pulse oximeter module 300 within the housing 201. The upper and lower housing 202, 203 are configured to pivot with the pulse oximeter module 300, so that the housing 201 opens and closes with the pulse oximeter module 300. The upper housing 202 and/or lower housing 203 may comprise a recess 205 at an end of the housing 201 opposite the finger receiving portion 204 to aid in opening the pulse oximeter 200. When the user presses down on the recess 205 on the upper housing 202, or elsewhere on the end of the upper housing 202, or squeezes the ends of the upper and lower housings 202, 203 together, the pulse oximeter module 300 contained within the housing 201 and the finger receiving portions 204, 301 open so that the user may use the pulse oximeter 200.

The pulse oximeter 200 of the present invention is designed to make the use of the pulse oximeter device 200 less stressful for the patient, by providing the pulse oximeter 200 with a more playful or relaxing structure.

A pulse oximeter module 300 is substantially enclosed within the housing. In certain embodiments of the invention, the pulse oximeter 200 may include a screen 206 configured to display data measured by the pulse oximeter 200 and other information pertinent to the operation of the pulse oximeter 200. The pulse oximeter 200 may also include an interface button 207 on the pulse oximeter 200 to enable the user to control operations of the pulse oximeter 200. In the embodiments of the pulse oximeter 200 shown in FIGS. 2A-3E, the pulse oximeter module 300 includes a screen 206 and an interface button 207 on the top of the pulse oximeter module 300 and the upper housing 202 comprises an opening 208 formed through the upper housing 202 to provide access to the screen 206 and interface button 207. The opening 208 in the upper housing 202 may include one opening 208 surrounding the screen 206 and interface button 207, or may comprise two separate openings 208 for each of the screen 206 and interface button 207, respectively. In alternative embodiments, such as the pulse oximeter 400 shown in FIGS. 4A-4D, the screen 406 and/or interface button 407 may be formed as part of the upper housing 402. In further alternative embodiments, the pulse oximeter may exclude a screen and/or interface button, and the upper housing may exclude the opening.

Various exploded views of the pulse oximeter 200 in accordance with an embodiment of the invention, including the housing 201 and pulse oximeter module 300, are shown in FIGS. 3A-3E.

The pulse oximeter module 300 may take the form of any pulse oximeter device known in the art, such as commercially available pulse oximeter devices or similar devices which are structurally modified for use with the housing 201. The pulse oximeter module 300 comprises the sensors and circuitry (not shown) for performing the measuring functionality and operations of the pulse oximeter as understood by those having ordinary skill in the art.

The pulse oximeter module 300 shown in FIGS. 3A-3E comprises a top chassis 302 and a bottom chassis 303 that are connected by a pivot 304. The top chassis 302 and bottom chassis 303 each comprise a groove 301a, 301b, and when the pulse oximeter module 300 is in its assembled state, the grooves 301a, 301b are arranged adjacently to form a finger receiving portion 301 of the pulse oximeter module 300, into which the patient can insert his or her finger to use the pulse oximeter 200. The top chassis 302 and bottom chassis 303 each comprise pivot receiving elements 304a, 304b, which are configured to align and receive a pin 304c of the pivot 304. The pivot 304 connects the top chassis 302 and bottom chassis 303 and enables the pulse oximeter module 300 to open and close when the end of the pulse oximeter module 300 opposite the finger receiving portion 301 is compressed and released. As discussed previously, when the user presses on or squeezes the end of upper and lower housing 202, 203, it causes the pulse oximeter module 300 to open.

The pulse oximeter module 300 may further include a battery holder 305 in the bottom chassis 303 configured to receive one or more batteries, for example two AAA batteries, for supplying power to the pulse oximeter module 300. The battery holder 305 may be accessed from the base of the bottom chassis 303 and a removable battery cover 306 provided to enclose the batteries. The user can therefore remove the lower housing 203 and the battery cover 306 to replace the batteries when needed. In alternative embodiments in which the housing 201 and pulse oximeter module 300 are integrally formed, the battery holder 305 and battery cover 306 may form part of the lower housing 203.

The pulse oximeter module 300 additionally may comprise a bezel 307 configured to receive the interface button 207 and screen 206. The bezel 307 comprises a screen receiving portion 308 and a button receiving portion 309 to receive the screen 206 and interface button 207, respectively. In certain embodiments, the screen receiving portion 308 and button receiving portion 309 may be separated and in other embodiments, the screen receiving portion 308 and button receiving portion 309 may be formed continuously as a single opening. Circuitry (not shown) for operating the interface button 207 and screen 206 may be arranged between the bezel 307 and the top chassis 302. The bezel 307 is configured to be attached to the top chassis 302 by, for example, screws, adhesive, a snap or compression fit, dowels or pins, or other connection means known in the art. In other embodiments of the invention, the bezel 307 and the top chassis 302 may be integrally formed.

In the embodiments of the pulse oximeter shown 200 in FIGS. 2A-3E, the housing 201 is configured to be removably attached to the pulse oximeter module 300. The housing 201 can therefore be removed in order to replace one housing 201 with an alternate housing 201. For example, multiple housings 201 having different designs can be supplied and can be exchanged for each other when the user so chooses. The housing 201 can also be removed in order to enable the replacement of batteries of the pulse oximeter module 300, as described previously.

In one embodiment, shown in FIGS. 3A-3E, the upper housing 202 is configured to be attached to a top surface of the pulse oximeter module 300 by attachment to the bezel 307 and/or top chassis 302 of the pulse oximeter module 300. An inner surface 209a of the upper housing 202 comprises a plurality of pins or dowels 210a that are configured to align with a plurality of holes 210b on the surface of the bezel 307. The upper housing 202 can be placed over the bezel 307, with the pins 210a and holes 210b aligned, and pressed down onto the bezel 307 such that the pins 210a are inserted into the holes 210b and the upper housing 202 is secured to the bezel 307 and the pulse oximeter module 300. The lower housing 203 is configured to be attached to a bottom surface of the pulse oximeter module 300 by attachment to the battery cover 306 and/or bottom chassis 303 in a similar manner. An inner surface 209b of the lower housing 203 comprises a plurality of pins or dowels 210c that are configured to align with a plurality of holes 210d on the surfaces of the battery cover 306 and/or bottom chassis 303. The lower housing 203 can be pressed onto the bottom chassis 303 with the pins 210c and holes 210d aligned, such that the pins 210c are inserted into the holes 210d and the lower housing 203 is secured to the bottom chassis 303 and the pulse oximeter module 300. In alternative embodiments, the arrangement of the pins 210c and holes 210d may be reversed, such that the pulse oximeter module 300 comprises the plurality of pins 210a, 210c and the upper and lower housings 202, 203 comprise the plurality of holes 210b, 210d to receive the pins 210a, 210c.

Other mechanisms for removably attaching the housing 201 to the pulse oximeter module 300 can be provided. For example, as shown in FIG. 2F, the top and/or bottom chassis 302, 303 can be provided with a locking tab 211a and the upper and/or lower housing 202, 203 provided with a corresponding tab receiving portion 211b (or vice versa), configured to receive and engage the locking tab 211a to secure the upper and/or lower housing 202, 203 to the pulse oximeter module 300. Alternatively, screws or bolts may be provided for attaching the upper and lower housing 202, 203 to the top and bottom chassis 302, 303, respectively.

In alternative embodiments of the invention, the housing 201 may be provided with means for removably attaching to the pulse oximeter module 300 that do not require any further structural elements on the pulse oximeter module 300. In this manner, the housing 201 can be provided for use in combination with any existing pulse oximeter device or module that can be purchased off the shelf. For example, the upper and lower housing 202, 203 may comprise one or more locking elements extending from one or both ends of the housing 201 that are configured to snap or latch onto the ends of the top chassis 302 and bottom chassis 303 of the pulse oximeter module 300. Adhesive materials may be provided for attaching the upper and lower housings 202, 203 to surfaces of the pulse oximeter module 300. The upper housing 202 and lower housing 203 may be dimensioned and made from a suitably pliable material so as to compress around the top and bottom chassis 302, 303 to provide a tight fit. Alternatively, compression elements, such as springs, can be provided on the ends of the inner surface 209a of the upper and lower housing 202, 203 and configured to engage the top and bottom chassis 302, 303 to secure the upper and lower housing 202, 203 to the pulse oximeter device 300.

In further alternative embodiments, the housing 201 of the pulse oximeter 200 shown in FIGS. 2A-2D may be formed integrally with the pulse oximeter module 300, such that the housing 201 is not a removable cover over the pulse oximeter module 300. For example, the upper housing 202 can be molded with or permanently affixed to the top chassis 302 of the pulse oximeter module 300 with the screen 206 and interface button 207 arranged therein or omitting the screen 206 and interface button 207 in certain embodiments. The lower housing 203 can be molded with or permanently affixed to the bottom chassis 303 of the pulse oximeter module 300. In such an embodiment, the pulse oximeter 200 is provided with fewer components to potentially assemble or disassemble.

In another embodiment of the present invention, shown for example in FIGS. 4A-4D, the specialized pulse oximeter housing 401 may be formed integrally with the pulse oximeter module. The three-dimensional housing 401 design elements may be molded with the top chassis and the bottom chassis so that the upper housing 402 is formed integrally with the top chassis and the lower housing 403 is formed integrally with the bottom chassis, rather than the upper and lower housings 402, 403 serving as detachable or removable covers for the pulse oximeter module as shown in FIGS. 2A-3E.

In the pulse oximeter 400 of FIGS. 4A-4D, the upper housing 402 and lower housing 403 are provided with three-dimensional surface elements to resemble a shark. In particular, the surface elements of the upper and lower housing 402, 403 are configured so that the finger receiving portion 404 of the pulse oximeter 400 resembles the mouth of a shark. The upper and lower housing 402, 403 are connected by way of pivots 405, so that the pulse oximeter 400 is configured to open and close in the manner previously described herein. The pulse oximeter 400 may comprise a screen 406 and/or an interface button 407 on the upper housing 402.

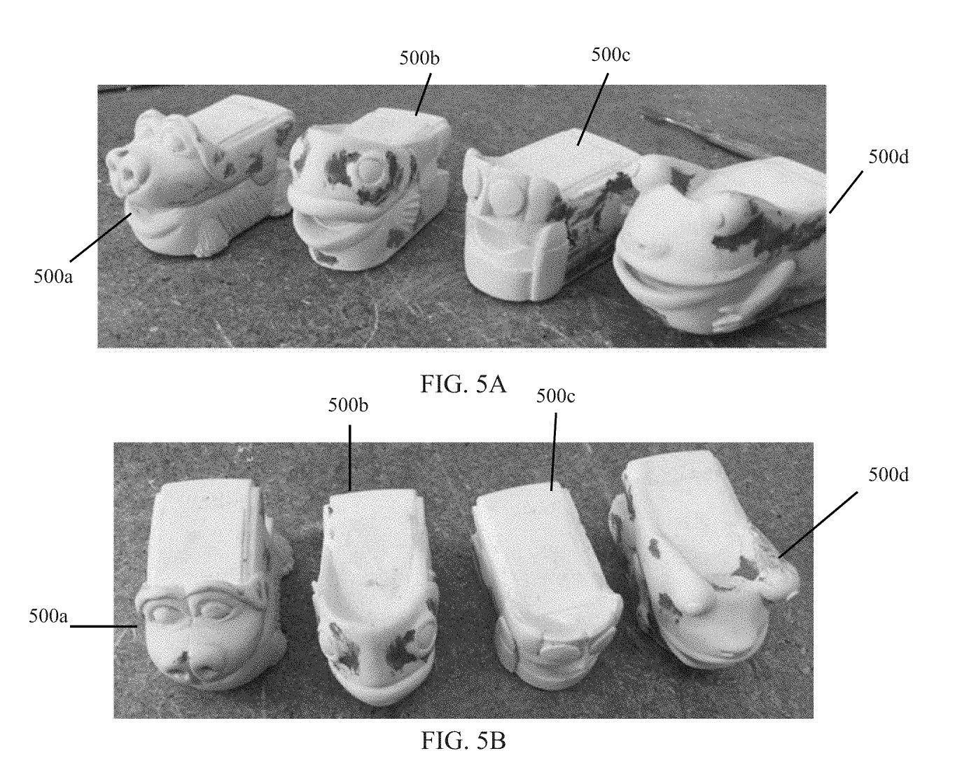

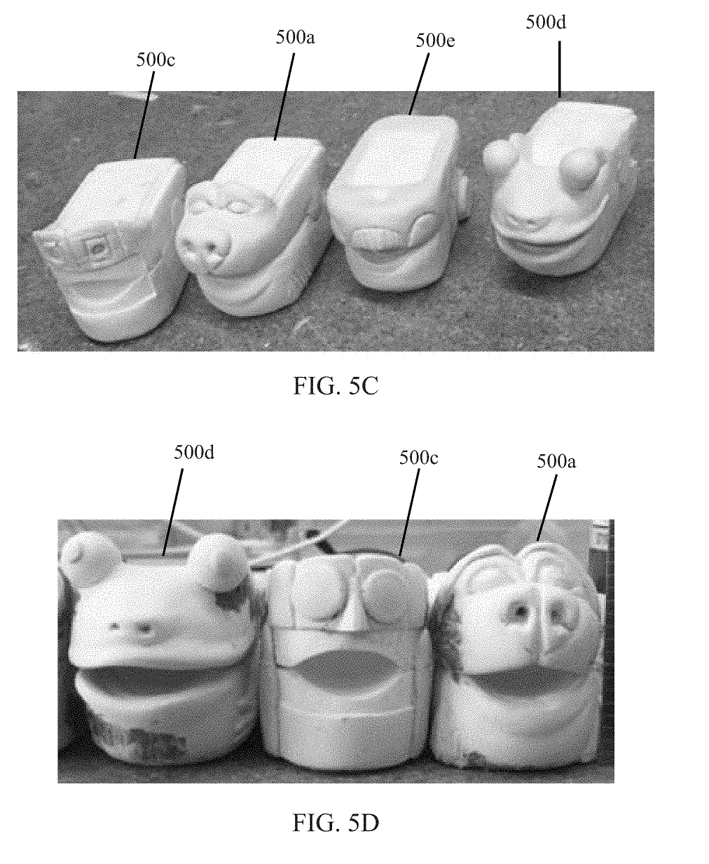

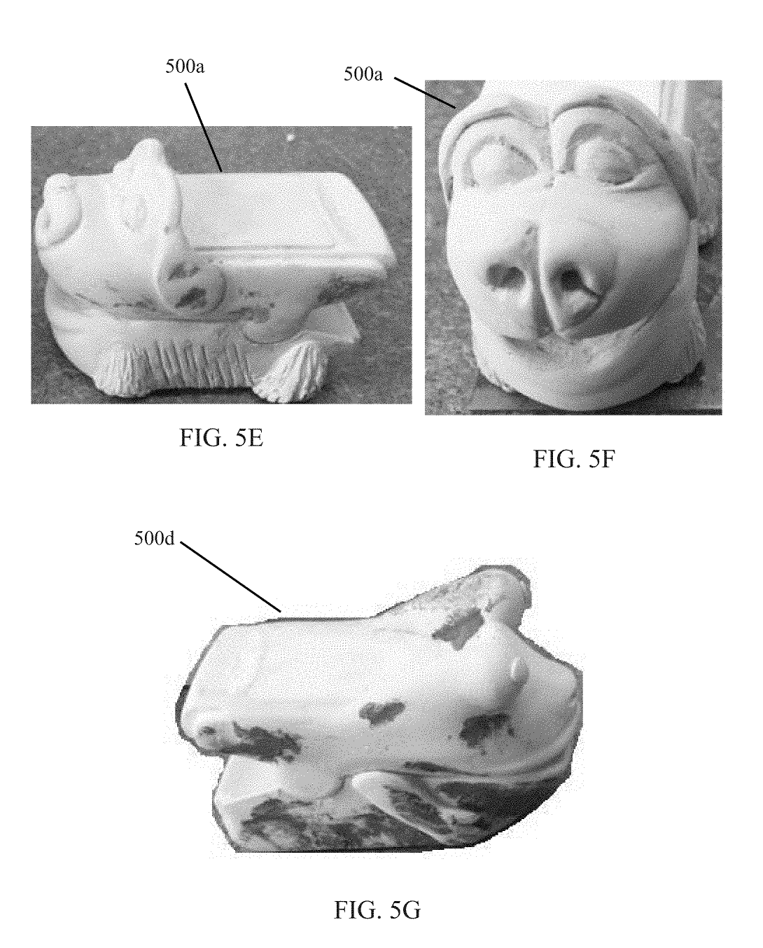

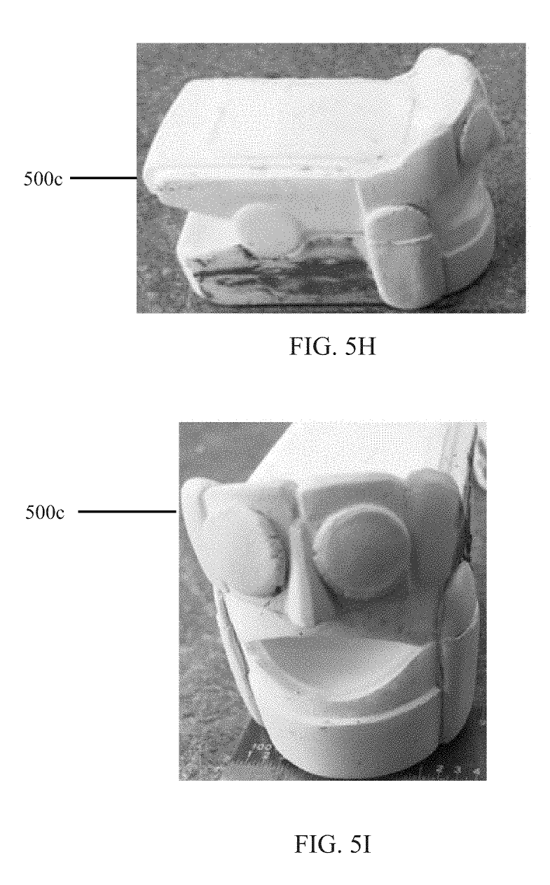

The pulse oximeter housing of the present invention may comprise three-dimensional surface elements to resemble any number of animals or other objects without departing from the scope of the invention. As shown in FIGS. 1-4D, pulse oximeter housings 101, 201, 401 may be provided that resemble an alligator, a fish or a shark, for example. FIGS. 5A-5I show further examples of pulse oximeter housings in accordance with the present invention configured to resemble a dog 500a, fish 500b, robot 500c, frog 500d or car 500e. Further housing designs may include, for example, a, duck, whale, koi or any other animal or object designed to have the appearance of a mouth around the finger receiving portion of the pulse oximeter. Other embodiments of the present invention may include housings or covers having additional animal types or shapes of objects that may not include a mouth. Additional designs may include, for example, ladybug, butterfly, dragonfly, a heart, football, and design shapes related to sports, film, TV and/or music, or other categories of intellectual property.

In alternative embodiments, the pulse oximeter can be also configured for use with and clipping onto a patient's ear without deviating from the scope of the invention.

The housing may also be configured to emit an audio sound, such as the sound of the animal that is presented in the housing design. For example, if the housing is designed to resemble a dog, the housing may comprise a noise generator, speaker and appropriate circuitry to emit a dog bark sound from the speaker when the housing is opened. In embodiments of the present invention in which the housing is in the design of an animal or other shape having eyes or eye-like elements, the eyes may be configured to light up, for example upon opening the pulse oximeter housing.

While there have been shown and described and pointed out fundamental novel features of the invention as applied to preferred embodiments thereof, it will be understood that various omissions and substitutions and changes in the form and details of the devices and methods described may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice.

What is claimed:

1. A pulse oximeter comprising:
a pulse oximeter module configured to measure pulse and oxygen saturation; and
a housing for the pulse oximeter comprising:
an upper housing arranged on a top portion of the pulse oximeter module comprising three-dimensional surface elements configured to resemble a first portion of an animal or an object; and
a lower housing arranged on a bottom portion of the pulse oximeter module comprising three-dimensional surface elements configured to resemble a second portion of the animal or the object.

2. The pulse oximeter according to claim 1, wherein the pulse oximeter module comprises a finger receiving portion configured to receive a finger of a user to measure pulse and oxygen saturation of the user, and
wherein the upper housing and lower housing define a housing finger receiving portion overlapping the pulse oximeter module finger receiving portion, and the three-dimensional surface elements of the upper housing and lower housing are collectively configured to resemble a mouth and a face of the animal or object for receiving the finger of the user into the pulse oximeter module finger receiving portion.

3. The pulse oximeter according to claim 1, wherein the upper housing is integrally formed with the top portion of the pulse oximeter module and the lower housing is integrally formed with the bottom portion of the pulse oximeter module.

4. The pulse oximeter according to claim 1, wherein the upper housing is configured to be removably attached to the top portion of the pulse oximeter module and to at least partially cover the top portion of the pulse oximeter module, and
wherein the lower housing is configured to be removably attached to the bottom portion of the pulse oximeter module and to at least partially cover the bottom portion of the pulse oximeter module.

5. The pulse oximeter according to claim 4, wherein the upper housing comprises an inner surface having a plurality of pins or dowels extending therefrom and a surface of the top portion of the pulse oximeter module comprises a plurality of holes configured to receive the plurality of pins or dowels of the upper housing to removably attach the upper housing to the top portion of the pulse oximeter module; and
wherein the lower housing comprises an inner surface having a plurality of pins or dowels extending therefrom and a surface of the bottom portion of the pulse oximeter module comprises a plurality of holes configured to receive the plurality of pins or dowels of the lower housing to removably attach the lower housing to the bottom portion of the pulse oximeter module.

6. The pulse oximeter according to claim 4, wherein the top portion of the pulse oximeter module comprises a top chassis and the bottom portion of the pulse oximeter module comprises a bottom chassis,
wherein the top chassis and the bottom chassis define a finger receiving portion therebetween configured to receive a finger of a user to measure pulse and oxygen saturation of the user, and
wherein the top chassis and the bottom chassis are attached by a plurality of pivots configured to enable the opening and closing of the pulse oximeter module to receive the finger of the user.

7. The pulse oximeter according to claim 6, wherein the pulse oximeter module further comprises:
a display screen;
an interface button; and
a bezel attached to the top chassis and configured to receive and house the display screen and the interface button.

8. The pulse oximeter according to claim 7, wherein the bottom chassis further comprises a battery holder configured to receive one or more batteries for supplying electric power to the pulse oximeter module and a removable battery cover configured to cover the battery holder.

9. The pulse oximeter according to claim 8, wherein the upper housing comprises an inner surface having a plurality of pins or dowels extending therefrom and the bezel comprises a plurality of holes configured to receive the plurality of pins or dowels of the upper housing to removably attach the upper housing to the bezel and top chassis of the pulse oximeter module; and
wherein the lower housing comprises an inner surface having a plurality of pins or dowels extending therefrom and the bottom chassis and the battery cover of the pulse oximeter module comprise a plurality of holes configured to receive the plurality of pins or dowels of the lower housing to removably attach the lower housing to the bottom chassis of the pulse oximeter module.

10. The pulse oximeter according to claim 1, wherein the three-dimensional surface elements of the upper housing and the three-dimensional surface elements of the lower housing are collectively configured to resemble an alligator, a fish, a shark, a dog, a car, a robot or a frog.

11. The pulse oximeter according to claim 1, wherein the top portion of the pulse oximeter module and the bottom portion of the pulse oximeter module are attached by a plurality of pivots configured to enable the opening and closing of the pulse oximeter to receive a finger or an ear of a user.

12. The pulse oximeter according to claim 4, wherein the upper housing comprises a locking tab and the top portion of the pulse oximeter module comprises a tab receiving portion configured to receive and engage the locking tab of the upper housing to removably attach the upper housing to the top portion of the pulse oximeter module; and
wherein the lower housing comprises a locking tab and the bottom portion of the pulse oximeter module comprises a tab receiving portion configured to receive and engage the locking tab of the lower housing to removably attach the lower housing to the bottom portion of the pulse oximeter module.

13. The pulse oximeter according to claim 4, wherein the top portion of the pulse oximeter module comprises a locking tab and the upper housing comprises a tab receiving portion configured to receive and engage the locking tab of the top portion of the pulse oximeter module to removably attach the upper housing to the top portion of the pulse oximeter module; and
wherein the bottom portion of the pulse oximeter module comprises a locking tab and the lower housing comprises a tab receiving portion configured to receive and engage the locking tab of the bottom portion of the pulse oximeter module to removably attach the lower housing to the bottom portion of the pulse oximeter module.

14. The pulse oximeter according to claim 4, wherein the upper housing and the top portion of the pulse oximeter module comprise overlapping openings configured to receive a screw or bolt to removably attach the upper housing to the top portion of the pulse oximeter module; and
wherein the lower housing and the bottom portion of the pulse oximeter module comprise further overlapping openings configured to receive a further screw or bolt to removably attach the lower housing to the bottom portion of the pulse oximeter module.

15. The pulse oximeter according to claim 4, wherein the upper housing is made from a flexible material and is configured to engage the top portion of the pulse oximeter module to removably attach the upper housing to the top portion of the pulse oximeter module; and
wherein the lower housing is made from a flexible material and is configured to engage the lower housing to removably attach the lower housing to the bottom portion of the pulse oximeter module.

16. The pulse oximeter according to claim 4, wherein the upper housing comprises one or more clip or tab extending from the upper housing that is configured to clip the upper housing to the top portion of the pulse oximeter module to removably attach the upper housing to the top portion of the pulse oximeter module; and
wherein the lower housing comprises one or more clip or tab extending from the lower housing that is configured to clip the lower housing to the lower housing to removably attach the lower housing to the bottom portion of the pulse oximeter module.

17. The pulse oximeter according to claim 3, wherein the wherein the upper housing is integrally molded with the top portion of the pulse oximeter module and the lower housing is integrally molded with the bottom portion of the pulse oximeter module.

18. The pulse oximeter according to claim 1, wherein the pulse oximeter module comprises an ear receiving portion configured to receive an ear of a user to measure pulse and oxygen saturation of the user, and
wherein the upper housing and lower housing define a housing ear receiving portion overlapping the pulse oximeter module ear receiving portion, and the three-dimensional surface elements of the upper housing and lower housing are collectively configured to resemble a mouth and a face of the animal or object for receiving the ear of the user into the pulse oximeter module ear receiving portion.

19. The pulse oximeter according to claim 1, wherein the pulse oximeter module is configured to emit an audio tone upon opening of the pulse oximeter, and
wherein the audio tone corresponds to a sound relating to the animal or the object resembled by the housing.

20. The pulse oximeter according to claim 1, wherein the pulse oximeter comprises one or more lighting elements configured to emit a light through the three-dimensional surface elements of either or both of the upper housing and lower housing.
